(19) United States
(12) Patent Application Publication
(10) Pub. No.: US 2008/0287884 A1
(43) Pub. Date: Nov. 20, 2008

(54) CONVERTIBLE SYRINGE SYSTEM

(76) Inventors: Matthew P. Warden, Boston, MA (US); Jeffrey P. Derderian, Billerica, MA (US); Suneer Chander, Chesnut Hill, MA (US); Ashley R. Winn, Somerville, MA (US)

Correspondence Address: GOODWIN PROCTER LLP;PATENT ADMINISTRATOR, EXCHANGE PLACE, BOSTON, MA, 02109-2881, US

(21) Appl. No.: 12/104,192

(22) Filed: Apr. 16, 2008

Related U.S. Application Data
(60) Provisional application No. 60/923,777, filed on Apr. 17, 2007.

(51) Int. Cl. A61M 5/315

(52) U.S. Cl. 604/220

(57) ABSTRACT

The present invention relates to a syringe system that allows rapid conversion between a needle/syringe system and a needleless/syringe system and in doing so allows an operator to move seamlessly between both systems, while minimizing needle stick risk. In this way, an operator may rapidly access medication bottles requiring a needle puncture and access needleless tubing systems requiring a needleless interface.

### CROSS-REFERENCE TO RELATED APPLICATION

This application claims priority to and the benefit of, Provisional Patent Application Ser. No. 60/923,777, filed on Apr. 17, 2007, the entire disclosure of which is hereby incorporated by reference herein.

### TECHNICAL FIELD

The present invention relates to a medical syringe for the withdrawal of medication from medication bottles and injection of medication into patients via medication tubing systems, and more particularly switching between a needle interface for withdrawal of medications from medication bottles and a needleless interface for injecting the medicine into a tubing system.

### BACKGROUND OF THE INVENTION

Currently, certain medical treatments require caregivers to place themselves at risk of needle stick, because of the requirements of working with needles and patient bodily fluids. Recent technological improvements have been safety focused and have lessened this risk with the trend toward needleless tubing systems and procedures involving retractable needles in catheters. Existing systems, however, continue to require caregivers to use a needle to draw medication from medication bottles and then convert this needle/syringe system to a needleless system by removing the needle. In addition to the safety concerns, caregivers must locate and open two different packages, attach the needle to the syringe, draw up medication, remove the needle, then attach the syringe to the needleless tubing system and inject the medication, and occasionally repeat the process.

One known system utilizes a plastic needle-like system that can both puncture a medication bottle and access tubing systems by a puncture of the tubing system hub. This system utilizes a sharp point, like standard needles, and must be pushed into the needleless port. Inherent in this action is the risk of missing the port and sticking yourself with the device. The avoidance of this risk is the primary driver for innovative products in safer needleless systems. Moreover, the puncture of needleless hubs with needles or pointed plastic pieces may result in the creation of a leak in the tubing system and allow backflow of fluid out of the punctured hub.

Some of the newer syringe devices focus on minimizing needle sticks by having a blunt cannula attached to the syringe that is used to puncture the bottle. The cannula is removed after the medication is drawn up and a sharp needle is attached. This device does add a layer of additional protection, but is inefficient, because the cannula must be removed and a needle attached.

A second problem with newer safety systems is their potential lack of intercompatibility. For example, a nurse may draw up the medication with one system for an intramuscular injection. If the physician changes her mind and orders the medication be given intravenously, the syringe holding the medicine may be incompatible with intravenous injection system.

### SUMMARY OF THE INVENTION

The present invention speeds the process, adds a layer of safety, and allows rapid conversion between a needle/syringe system and a needleless/syringe system, thus allowing an operator to move seamlessly between both systems while minimizing needle stick risk. In particular, the current invention provides for the insertion of the syringe into the needleless system with no exposed sharp point that may inadvertently lead to a skin puncture.

The system allows for a collar or sheath with an attached fluid port to slide over the fixed needle at the end of the syringe and reversibly lock in an advanced position creating a functionally needleless adaptor for interfacing with the needleless tubing systems. The system also allows for retraction of the collar to expose the fixed needle at the end of the syringe for puncturing medication bottles.

In use, the operator starts with the collar in a retracted position such that the needle is exposed, which allows the operator to puncture a medication bottle with the needle of the syringe. The operator then pulls back on the syringe plunger drawing medication through the needle and into the barrel of the syringe. When the correct amount of fluid has been drawn into the syringe barrel, the needle is withdrawn from the medication bottle. The operator then slides the collar forward and locks it in an advanced position that simultaneously covers the end of the needle and presents a needleless fluid port (e.g., a luer lock fitting) for interfacing with a tubing system. The operator can then connect the syringe to the needleless tubing system (e.g., by screwing the luer lock fitting to a corresponding fitting on the tubing system) and administer the medication by advancing the syringe plunger.

In one aspect, the invention relates to a syringe system including a syringe barrel defining an interior space, a syringe plunger slidably disposed within the syringe barrel, a needle coupled to a distal end of the syringe barrel and in fluid communication with the interior space, a collar slidably disposed about the syringe barrel, and a fluid port disposed at a distal end of the collar. The collar can be coupled to the syringe barrel to prevent inadvertent disengagement therefrom. The fluid port is configured to interface with a needleless tubing system.

In another aspect, the invention relates to a syringe system including a syringe barrel defining an interior space, a syringe plunger slidably disposed within the syringe barrel, a needle coupled to a distal end of the syringe barrel and in fluid communication with the interior space, a collar threadedly engaged with the syringe barrel, and a fluid port disposed at a distal end of the collar, the fluid port configured to interface with a needleless tubing system. The collar can be moved along the length of the syringe barrel by rotational movement thereof.

In various embodiments of the foregoing aspects, the collar is configured to slide longitudinally along at least a portion of the syringe barrel. The collar can be secured in a first, retracted position exposing the needle and a second, advanced position encapsulating the needle. In the advanced position, the fluid port is presented for interfacing with the needleless tubing system. The fluid port can include threads and be secured to the needleless tubing system by a screw action (i.e., rotation of the port, collar, and/or syringe). In one embodiment, the fluid port is a luer lock type fitting, either male or female to suit the particular application. In addition, the collar can be locked in at least one of the first position and the second position, either reversibly or irreversibly. The syringe system can include a locking mechanism disposed on at least one of the syringe barrel and the collar for locking the collar in the first, retracted position and/or the second, advanced position. In one embodiment, the collar only locks in the advanced position. Additionally or alternatively, the collar can be locked in place by, for example, frictional engagement or with the use of an O-ring.

In additional embodiments of the syringe system, the syringe barrel can include at least one slotted rail disposed on an outer surface of the syringe barrel and oriented longitudinally along a length of the syringe barrel and the collar can include at least one protuberance disposed on an inner surface of the collar for engaging the slotted rail. The collar is configured to slide along the syringe barrel via engagement of the protuberance and the slotted rail. In one embodiment, two longitudinal rails can be disposed on the syringe barrel on opposite sides thereof, e.g., 180 degrees apart on a cylindrically shaped syringe barrel. Alternatively, more that two rails can be included depending, for example, on the size of the syringe. In one embodiment, the longitudinal rail includes a transverse portion extending from a distal end of the longitudinal rail. The transverse portion of the slotted rail allows guided, rotational movement of the collar when in the second position. Alternatively or additionally, the longitudinal rail can include a transverse portion extending from a proximal end of the longitudinal rail to provide guided, rotational movement of the collar when in the first position.

Furthermore, the locking mechanism can be disposed on the transverse portion of the rail and the collar engages the locking mechanism when rotated in the second position. In one embodiment, at least one of the transverse portions of the slotted rail includes a locking mechanism for securing the collar in at least one of the first position and the second position when the collar is rotated, such that the protuberance engages the locking mechanism. In another embodiment, the syringe system includes a second locking mechanism disposed near the proximal end of the longitudinal rail. The second locking mechanism configured to secure the collar in the first position. The locking mechanism in one embodiment can reversibly lock the collar in its position by frictional engagement between the protuberance and the rail. In another embodiment, the locking mechanism includes a projection that prevents the return rotational movement of the collar by blocking the protuberance. For example, the locking mechanism can be an inclined block that allows the protuberance to slide over the inclined surface, but is blocked by a vertical wall of the block when the operator attempts to rotate the collar into an unlocked position.

In still further embodiments of the syringe system, the collar is guided between the first position and the second position by a thread disposed on an external diameter of the syringe barrel, for example as opposed to sliding on the rails. The fluid port can be configured for attachment of a secondary needle, for example, by threaded engagement. The secondary needle can provide a different needle configuration and/or size for a particular application and can be attached to the syringe in its extended position. In addition, the collar can be biased in at least one of the first position and the second position to prevent the inadvertent movement of the collar when in use. In one embodiment, the collar includes a spring mechanism for biasing the collar in the second position. For example, the spring mechanism can be a spring or other resilient member disposed within the collar between a distal end of the syringe barrel and a distal interior end of the collar, thereby biasing the collar in the second, advanced position. The collar can be forced back against the spring to the first, retracted position and locked in place. Such an arrangement will fail safe with the collar in the advanced position, thereby covering the needle.

Moreover, the fluid port may conform to at least one of ISO standard 594-1 and 594-2. The collar can be removable from the syringe barrel and interchangeable with a second collar with an alternative fluid port. This arrangement allows the syringe assembly to be customized for the particular needleless tubing system to which it will interface. In one embodiment, the collar can be snap fit onto the syringe barrel and can be removed by flexing the collar such that the protuberance(s) are moved out of the slotted rail(s). Once the protuberances clear the slotted rail, the collar can be rotated and slid off of the syringe barrel. In another embodiment, a distal portion of the collar can include an internal channel for receiving at least a distal portion of the needle. The channel can be formed in the distal end of the collar or can include an additional cylindrical body formed coextensively with the collar and defining the channel running therethrough. The needle can be moved through the channel when the collar is retracted into the first position. The collar can further include an O-ring disposed in an annular groove formed in an internal surface of the channel. The O-ring provides a seal between an outer diameter of the needle and an inner diameter of the channel for the passage of a fluid between the syringe barrel and the needleless tubing system, without leakage. The seal facilitates generating a suction at the distal end of the collar when the collar is in the extended position and the syringe plunger is drawn back to draw fluid into the syringe barrel. Generally, the syringe plunger can be extended and retracted to draw in and dispense fluid, respectively, through the distal end of the collar through the fluid port between the syringe and the needleless system. In one embodiment, the friction between the O-ring and the needle will prevent or at least impede movement of the collar. Additionally, in an embodiment where the needle expands at its base, the increased interference fit between the outside diameter of the needle and the inside diameter of the O-ring will further secure the collar in place.

In yet another embodiment of the syringe system, the syringe barrel has a generally circular cross-sectional shape and the collar has a generally elliptical internal cross-sectional shape, which provides for a slight interference between the outside diameter of the syringe barrel and the inside surface of the collar. This arrangement can provide sufficient surface tension between the collar and syringe barrel to prevent sliding movement between the collar and the syringe barrel. A light pressure can be applied to the collar to deform the internal cross-sectional shape of the collar from generally elliptical to generally circular and of a larger diameter than the outside diameter of the syringe barrel to enable slidable movement therebetween. For example, the operator applies a squeezing force to the collar with one hand to deform the collar and initiates sliding of the collar with the other hand, thereby sliding the collar along the syringe barrel between the first and second positions. When the force is removed, the collar returns to the generally elliptically shaped internal cross sectional shape which provides a partial interference fit with the outside diameter of the syringe barrel, which provides sufficient surface tension to prevent movement of the collar.

These and other objects, along with advantages and features of the present invention herein disclosed, will become apparent through reference to the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1A is a schematic plan view of a syringe system in a retracted or needled position in accordance with one embodiment of the invention, the syringe system including a syringe barrel, a plunger, a needle, and a sliding collar;

FIG. 1B is a schematic plan view of the syringe system of FIG. 1A, with the collar and plunger removed;

FIG. 2 is a schematic plan view of the syringe assembly of FIG. 1A in partial cross-section, with the collar in an advanced position such that the needle is covered and a fluid port on the collar may engage a needleless system;

FIG. 3 is a schematic enlarged view of the fluid port portion of the collar of the syringe system of FIG. 2;

FIG. 4A is an enlarged schematic cross-sectional view of the collar of FIGS. 1-3;

FIG. 4B is an enlarged schematic view of the distal end of the barrel of FIGS. 1-3 rotated 90 degrees;

FIGS. 5A and 5B are schematic perspective views of a syringe system in accordance with one embodiment of the invention;

FIG. 6 is a schematic exploded view of a syringe system in accordance with one embodiment of the invention;

FIGS. 7A and 7B are schematic cross-sectional views of a syringe system in accordance with one embodiment of the invention with a spring that biases the collar and fluid port into an advanced position;

FIGS. 8A and 8B are schematic cross-sectional views of a syringe system in accordance with one embodiment of the invention with a collar that may be directed over the needle by a side-arm to secure the collar in the advanced position;

FIGS. 9A and 9B are schematic cross-sectional views of a syringe system in accordance with one embodiment of the invention with a threaded luer lock collar in a retracted position and in an advanced position;

FIGS. 10A and 10B are schematic cross-sectional views of a syringe system in accordance with one embodiment of the invention with a deformable elliptical collar in a locked and an unlocked position; and

FIGS. 11A and 11B are schematic cross-sectional views of an alternative embodiment of the syringe system of FIGS. 9A and 9B.

### DETAILED DESCRIPTION OF THE INVENTION

In the following, embodiments of a syringe system in accordance with the invention are further described with reference to a single application. It is, however, to be understood that the present invention can also be used for other types of syringe or needle systems. For example, in one embodiment, the syringe is a hypodermic syringe used with a hypodermic needle to inject liquid or gases into body tissues, or to remove liquid or gases from the body.

It will, therefore, be understood that the present invention is directed to a syringe for administering different fluids, which overcomes the problems of the prior art. In particular, a syringe system in accordance with the present invention includes a unique collar with a fluid port that may reversibly lock in a position to expose a puncturing needle or advance to shield the needle and provide a connection for needleless attachment to tubing systems. Manufacture of the component parts of the syringe of the present invention does not involve complicated and expensive manufacturing techniques or precise control over the dimensions of the component parts of the device.

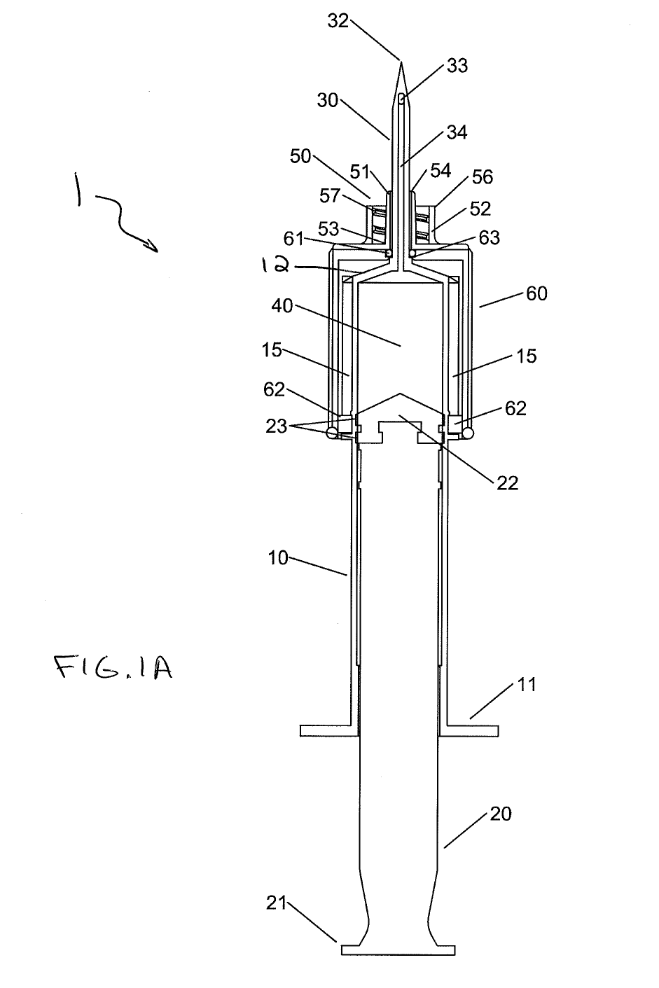

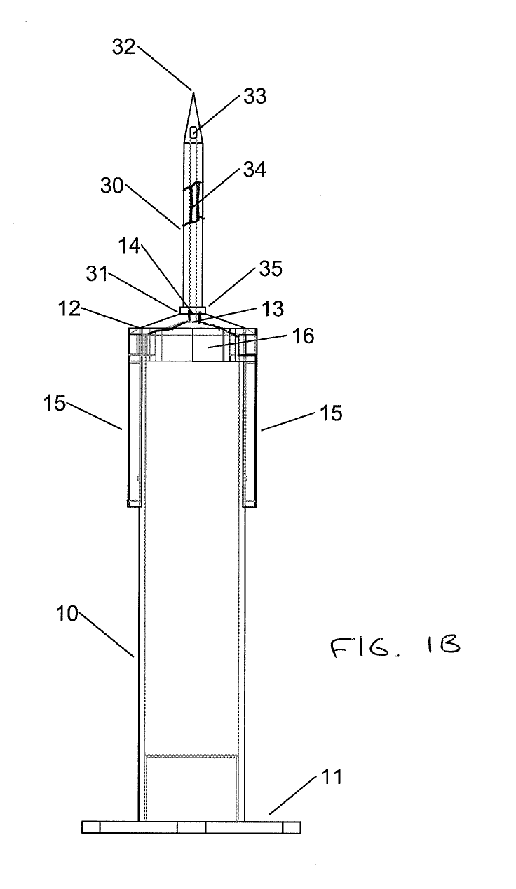

Referring now to the drawings, in particular FIGS. 1A and 1B, a disposable medical syringe system 1 in accordance with the invention includes a syringe barrel 10, in the form of a hollow cylinder defining an interior space 40. The system 1 further includes a plunger 20 inserted into a proximal end 11 of the syringe barrel 10. The plunger 20 can be advanced into the barrel 10 such that a distal rubber portion 22 of the plunger 20 contacts an internal distal end 12 of the barrel 10. The plunger 20 has a proximal handle 21 that is used by an operator to slide the plunger 20 through the interior space 40 of the barrel 10 from the proximal end 11 to the distal end 12 of the barrel 10. The plunger 20 includes one or more sealing surfaces 23 that provide a water tight seal between the distal rubber portion 22 of the plunger 20 and the internal wall of the barrel 10.

The barrel 10 includes a generally centrally located opening 13 formed in the distal end 12 of the barrel 10 through which fluid or medication may be pushed or pulled by actuating the plunger 20 and creating forces (e.g., pressure and vacuum) within the interior space 40, due to the watertight seal of the plunger 20 within the barrel 10. A hollow needle 30 is coupled to the distal endpoint 14 of the barrel 10 and is in fluid communication with the opening 13 and the interior space 40. The needle 30 has a proximal end 31 coupled to the distal endpoint 14 of the barrel 10 and a tapered distal end 32. The tapered distal end 32 of the needle 30 has two side ports 33 that allow fluid transfer and are disposed on the tapered distal end 32 of the needle 30.

The volume of the interior space 40 is variable and defined as the space within the barrel 10 and distal to the rubber portion 22 of the plunger 20. The volume of the space 40 can be varied by movement of the plunger 20, for example, moving the plunger proximally toward the proximal end 11 enlarges the volume. Movement of the plunger 20 toward the distal end 12 via operator action at the handle 21 causes medication or fluid to be moved by the distal end 22 of the plunger 20 toward the distal needle tip 32. For example, the fluid is driven through opening 13 and an internal channel 34 of the needle 30 out the needle's distal side ports 33.

Referring to FIGS. 1-3, the syringe system 1 includes at its distal end a collar 60 and a fluid port 50 disposed in the distal end of the collar 60. In one embodiment, the fluid port 50 is a luer lock type fitting. The collar 60 is coupled to the syringe barrel 10. In one embodiment, the luer lock 50 includes a longer internal cylinder 51 and a shorter wider surrounding cylinder 52. The cylinders 51, 52 extend from a distal end 53 of the collar 60, with the internal cylinder 51 extending beyond the surrounding cylinder 52 to a distal point 54. The internal cylinder 51 has an inner diameter (ID1) and an outer diameter (OD1). The surrounding cylinder 52 extends to a distal end 56 and has an inner diameter (ID2) and an outer diameter (OD2). The second inner diameter (ID2) of fluid port 50 includes an internal thread 57 molded therein. In one embodiment, the disposable medical syringe system 1 interfaces with a needleless tubing system via the circumferential thread 57.

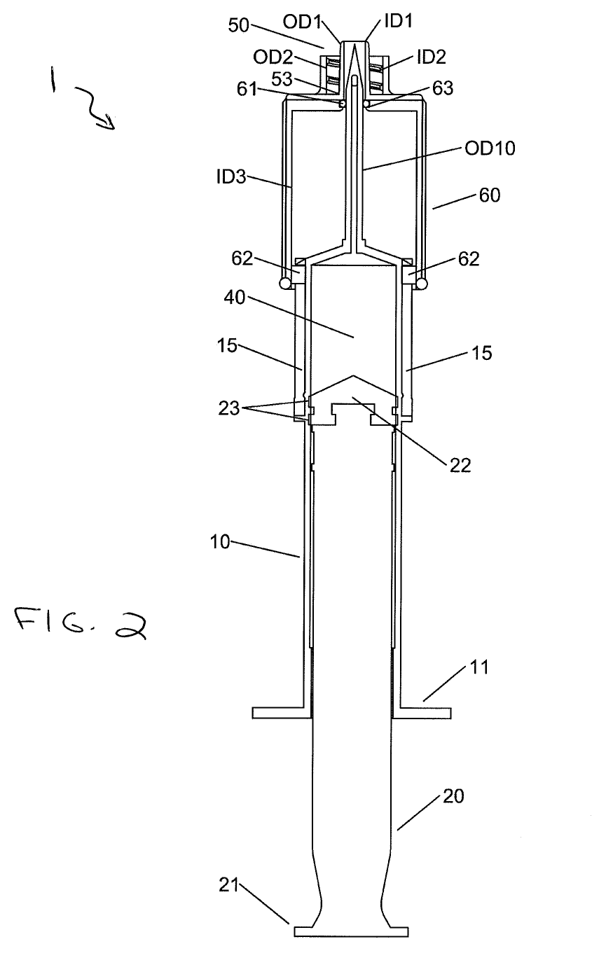

The collar 60 surrounds and is coupled to at least a portion of barrel 10 near its distal end 14. The collar 60 may slide back and forth along a portion of the barrel 10 such that when it is in the fully extended position (see FIG. 2), the distal end 32 of the needle 30 is covered by the distal end 54 of the fluid port 50. Alternatively or additionally, the collar 60 may be retracted over the barrel 10 as shown in FIG. 1A, resulting in full exposure of the needle 30.

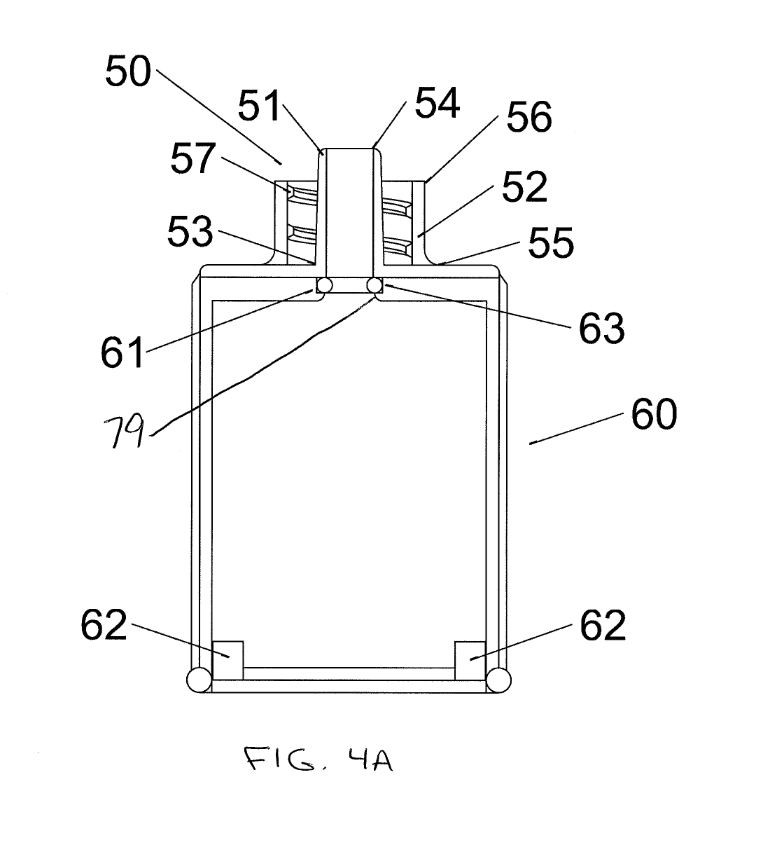

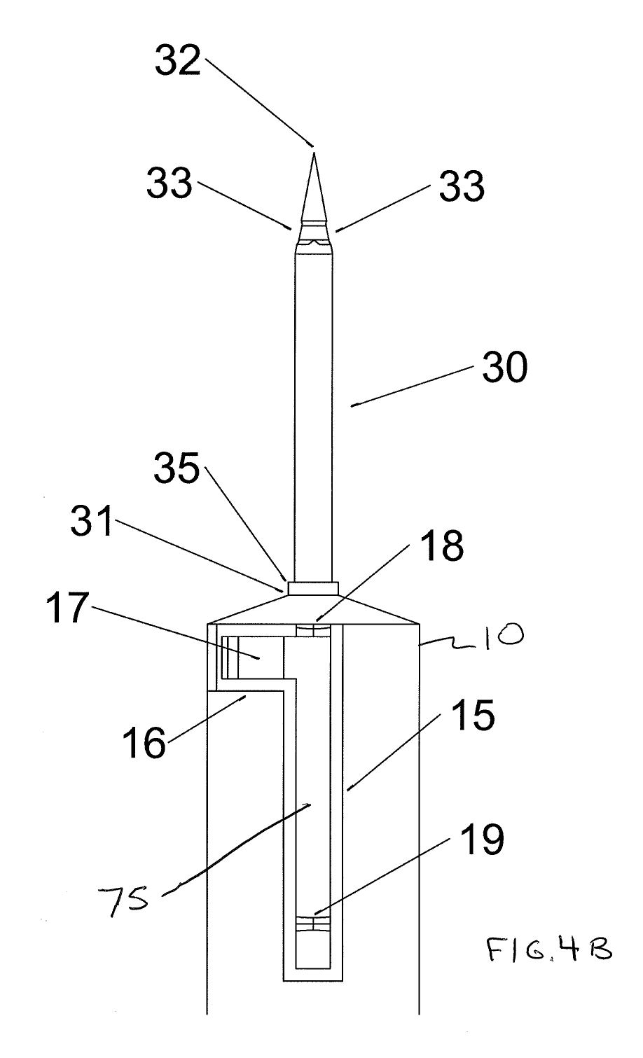

Referring to FIG. 1B, two slotted rails 15 are longitudinally disposed on an exterior surface of the barrel 10; however, more rails could be provided. In the embodiment shown, the two rails 15 are equally spaced about the circumference of the barrel 10. Referring to FIGS. 2 and 4A, the collar 60 includes two protuberances or fingers 62 extending from an interior surface (ID3) of the collar 60. The protuberances 62 engage the slots 75 (FIG. 4B) in the rails 15, thereby controlling the sliding motion of the collar 60 relative to the barrel 10 between the first, retracted position (FIG. 1A) and the second, extended position (FIG. 2). The barrel 10 may include a transversely extending side rail 16 that extends along at least a portion of the circumference of the barrel 10. The side rail 16 extends from a distal end of the slotted rail 15 and also is slotted for accommodating the protuberance 62. When the collar 60 is advanced to the extended position, the collar 60 can be rotated such that the protuberances 62 slide within the transverse side rails 16. By rotating the collar 60, it can be locked in place to prevent it from sliding back into its retracted position. In the embodiment shown in FIG. 4B, a locking mechanism 17 is disposed in the side rail 16 to prevent the protuberances 62 on the collar 60 from inadvertently rotating out of the side rails 16. In one embodiment, the locking mechanism is a ramp or inclined block that permanently locks the collar 60 in its extended position. Alternatively, the locking mechanism can be thinned area of the slotted side rail 16 that provides for frictional resistance to the movement of the protuberance 62. As shown in FIG. 4B, the rail 15 includes a locking mechanism 19 in the form of a bump or thinned area of the rail which provides enough frictional resistance to prevent inadvertent sliding of the collar 60 from the retracted position.

In one embodiment, again referring to FIG. 4B, the collar may be reversibly locked in the retracted or extended position when the sliding collar thread pushes through an inclined angle 19, 17 on the barrel located at the proximal and/or distal portion of the slotted rails 15, 16.

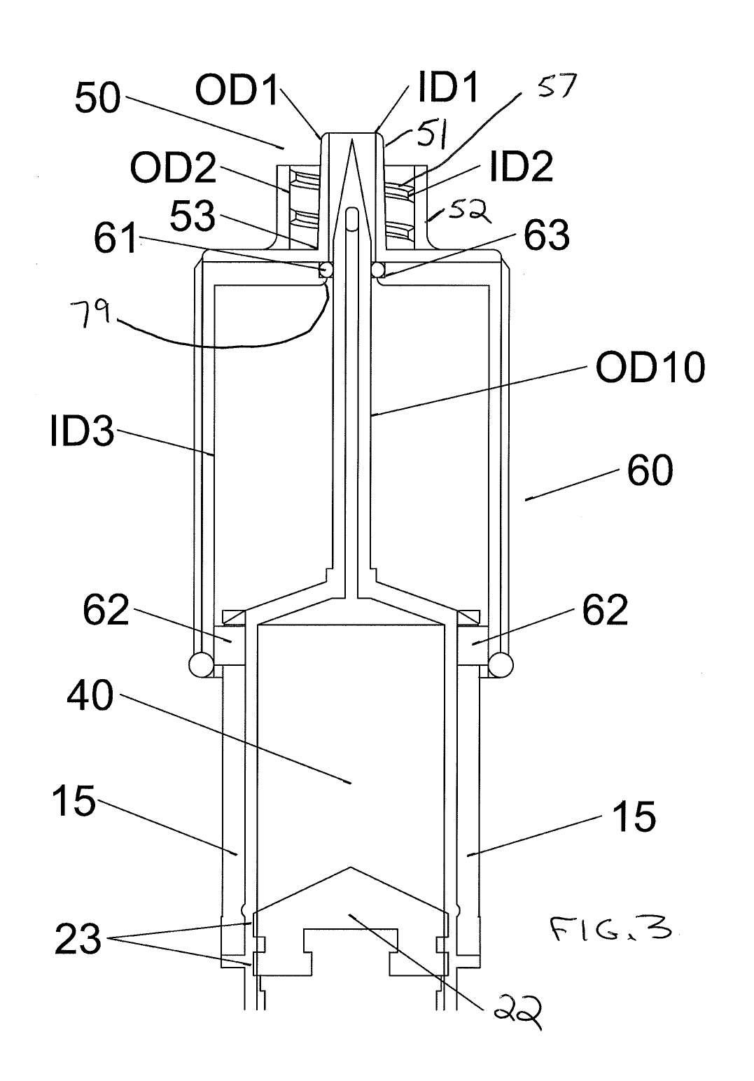

Referring to FIGS. 1A and 4A, the collar 60 includes a sealing component 61. The sealing component 61 can be a rubber o-ring disposed in an annular groove 63 formed in the inside diameter (ID1) of the fluid port. The sealing component 61 maintains a constant watertight seal between the internal diameter (ID1) of the fluid port 50 and the outer diameter (OD10) of the needle 30 through any movement of the collar 60 either proximally to expose the distal end 32 of needle 30 or distally to cover the distal end 32 of the needle 30 (see FIG. 3). This results in the prevention of any fluid leakage between the needle 30 and the collar 60. The system 1 may be attached to a needleless tubing system by the fluid port 50 and medication or other fluid may be injected from the interior space 40 through the needle 30 and out of the fluid port 50 and into the needleless tubing system. In one embodiment, the collar 60 includes an internal channel 79 passing through the distal end of the collar 60 and in which the groove 63 and O-ring 61 are disposed.

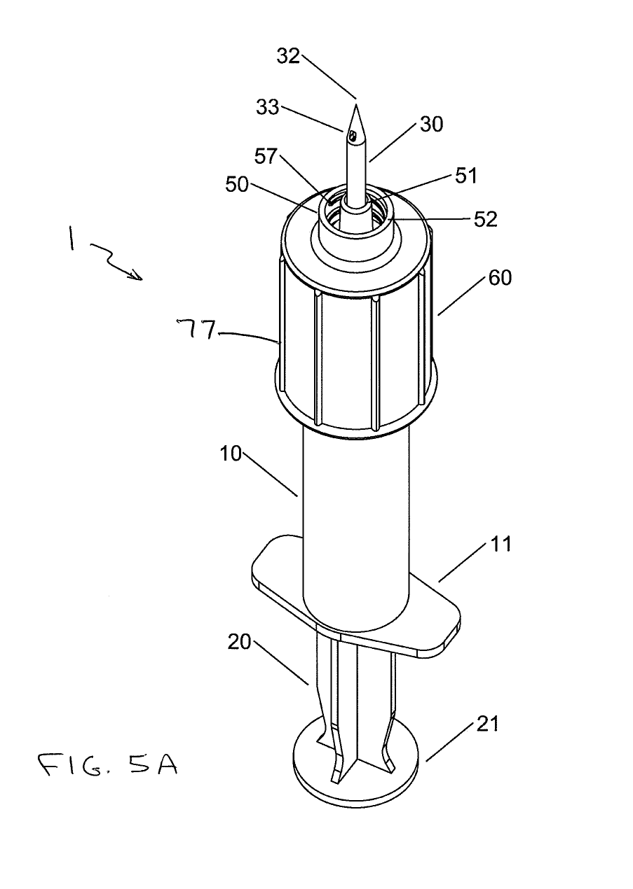

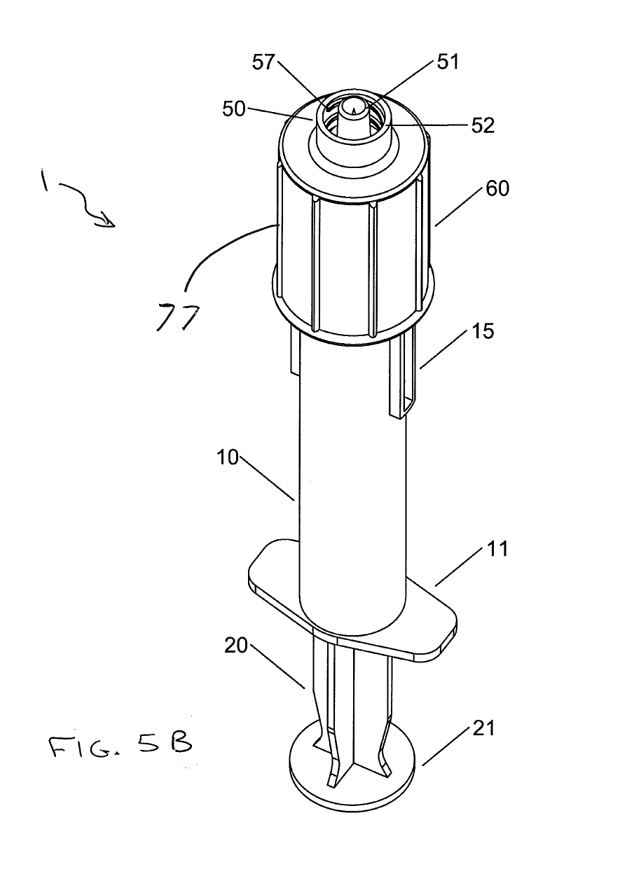

FIGS. 5A and 5B are perspective views of one embodiment of the invention from a user's vantage point. In FIG. 5A, the collar is in the retracted position. In FIG. 5B, the collar 60 is in the extended position. As shown, the collar 60 can include structure 77, such as knurling or protuberances that aid in the movement of the collar 60.

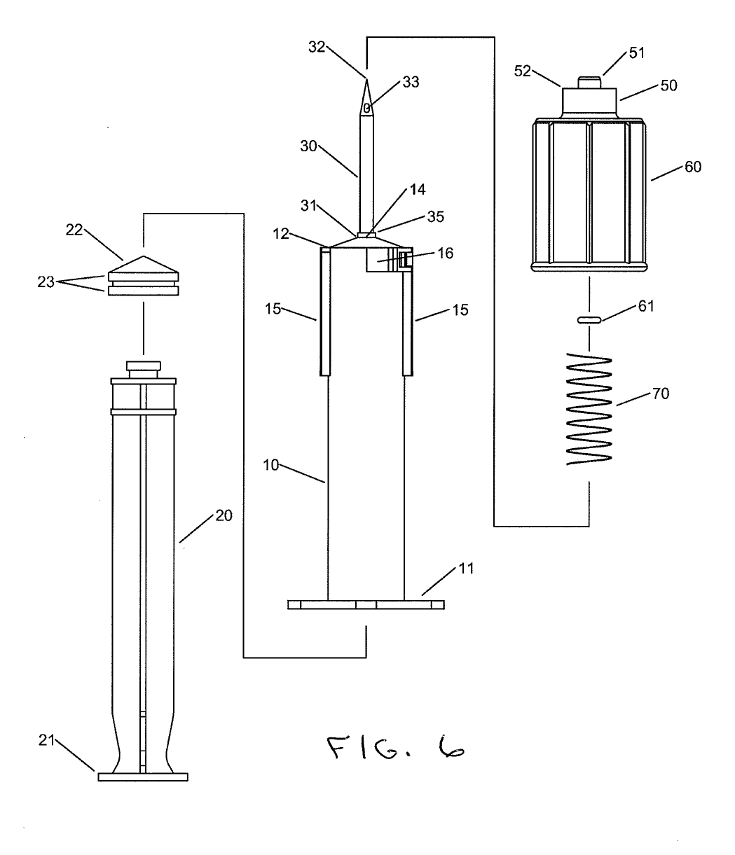

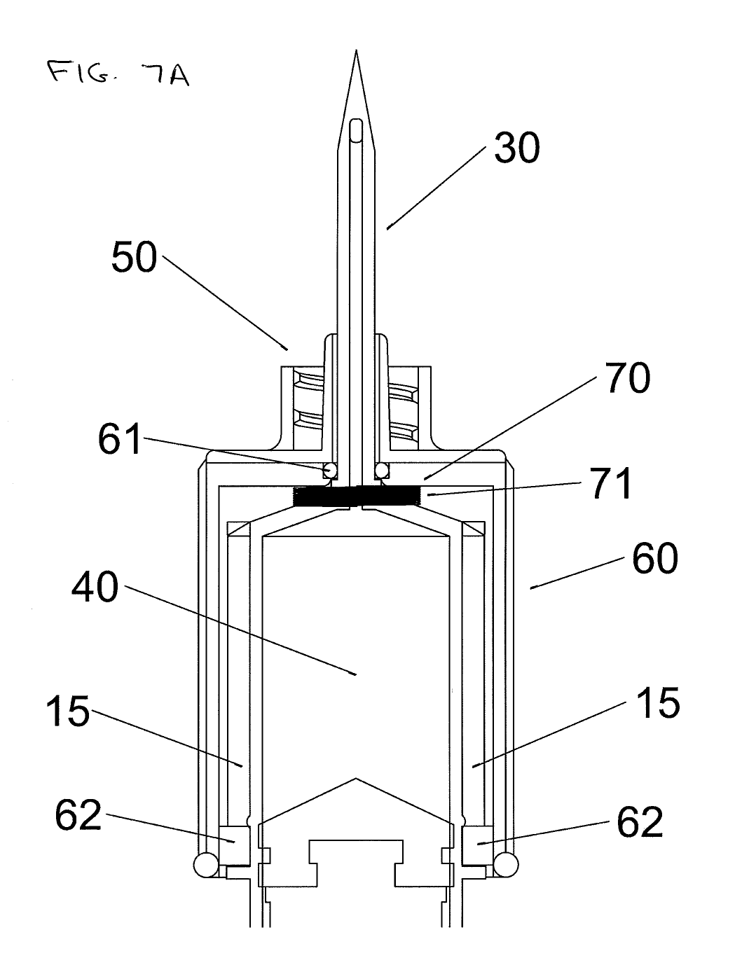

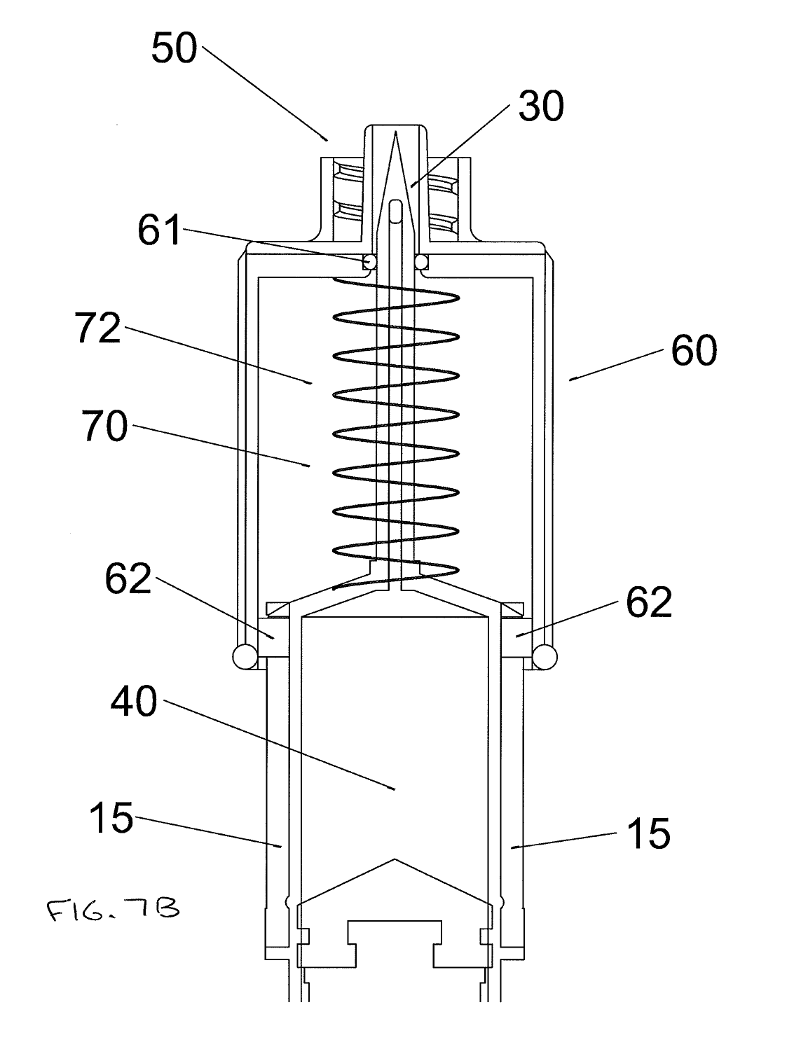

Additional embodiments include the use of a spring 70 (FIG. 6) that favors the uncoiled position 72 (biasing the collar into the advanced position as shown in FIG. 7B) over the coiled position 71 (i.e., the retracted position shown in FIG. 7A). The spring 70 can be secured against the internal surface of the distal end 53 of the collar 60 and the external surface of the distal end 12 of the barrel 10. These surfaces can also include grooves for holding the spring 70 in place.

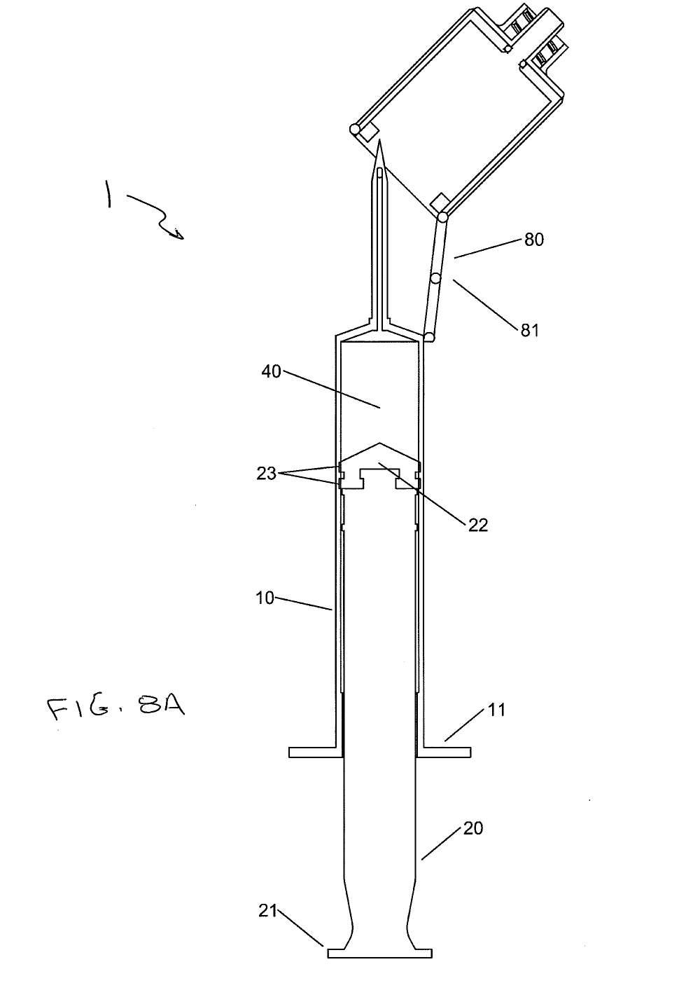

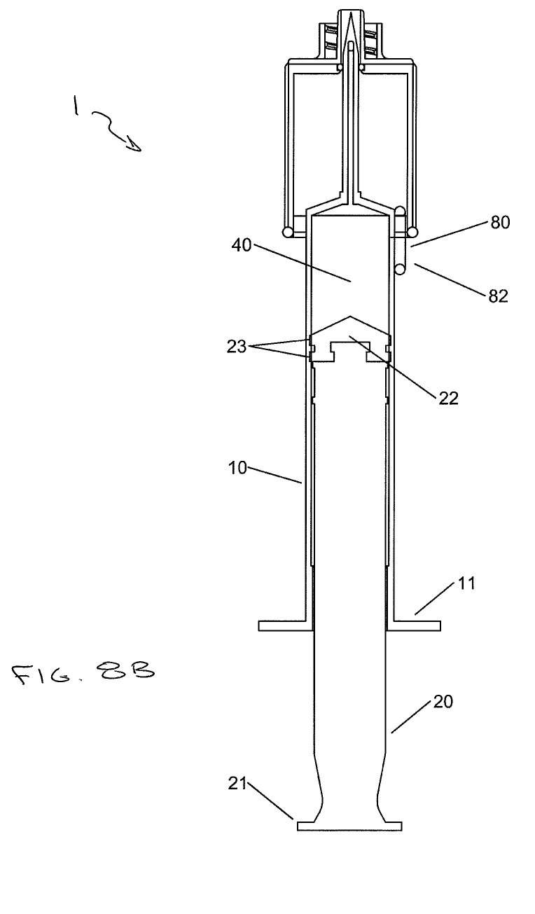

Another embodiment allows for a side action luer lock collar attachment as shown in FIGS. 8A and 8B. The side arm 80 is extended in position 81 and the collar 60 is held out to the side such that needle 30 can be used to draw up fluid. In FIG. 8B, the collar 60 is slid over the needle 30 and the side arm 80 is moved into a shortened position 82, such that the collar 60 covers the needle 30 and the luer lock can engage needleless tubing systems. The arm 80 can be a linkage or other mechanical assembly that can move the collar 60 between the two positions and may include structure for securing the collar 60 in at least one of the two positions, for example the advanced position on the syringe barrel 10.

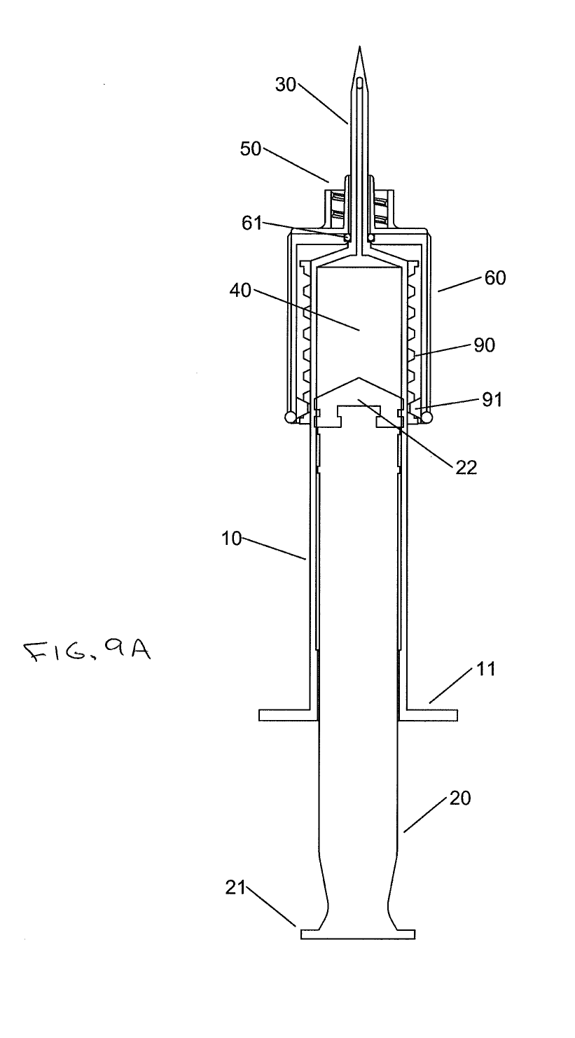

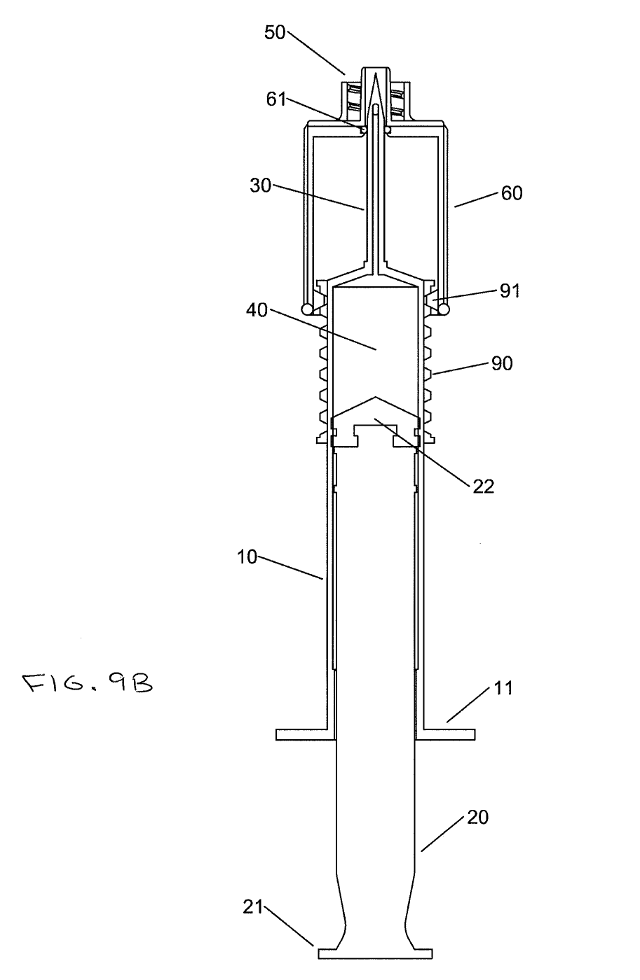

In one embodiment (FIGS. 9A and 9B), the rails are replaced by a thread 90 running around the barrel and the collar has a protuberance 91 which may be locked in a retracted position (FIG. 9A) or the advanced position (FIG. 9B) by rotation of the collar around the barrel. The collar 60 may be locked in place by frictional forces between the thread 90 and protuberance 91.

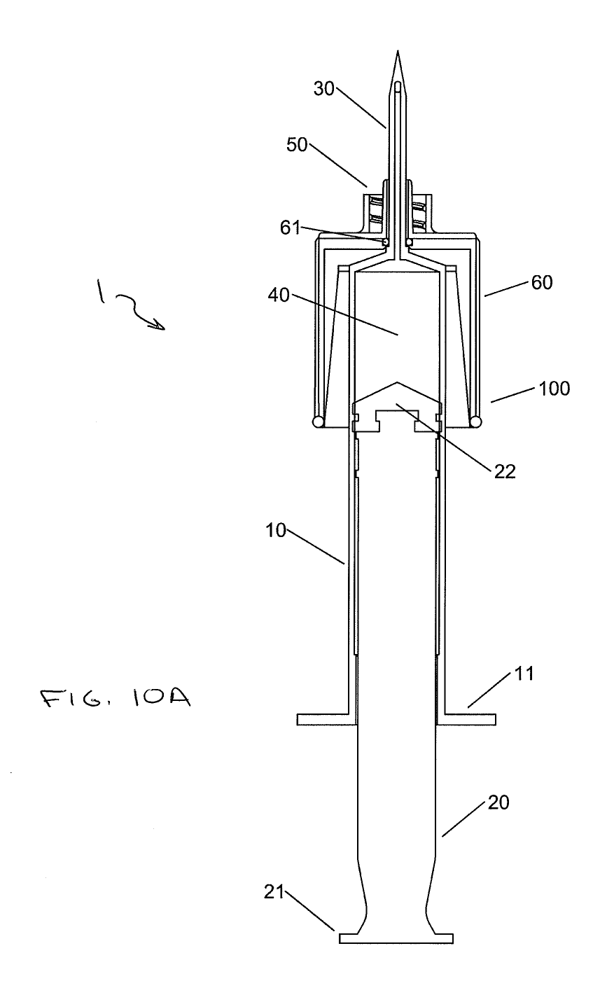

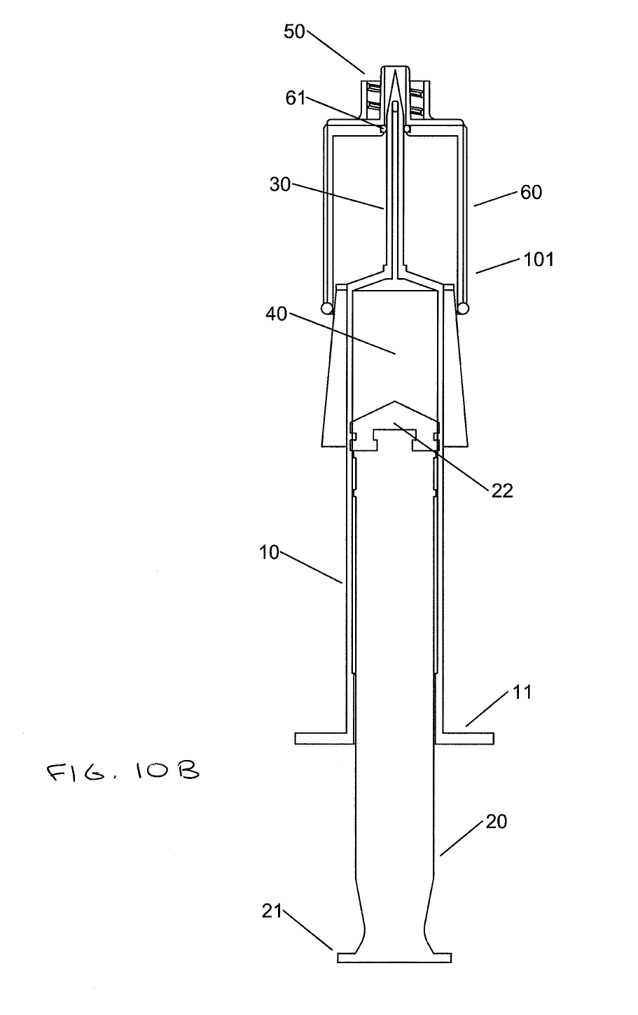

In another embodiment (FIGS. 10A and 10B), the collar 60 has an elliptical or circular circumference in the retracted position (elliptical and locked in FIG. 10A) which is then deformed to the other shape (elliptical or circular) to move the collar into the advanced position (circular and slideable in FIG. 10B) to aid in reversibly holding the collar in the retracted or advanced position by the frictional engagement of the collar with the barrel.

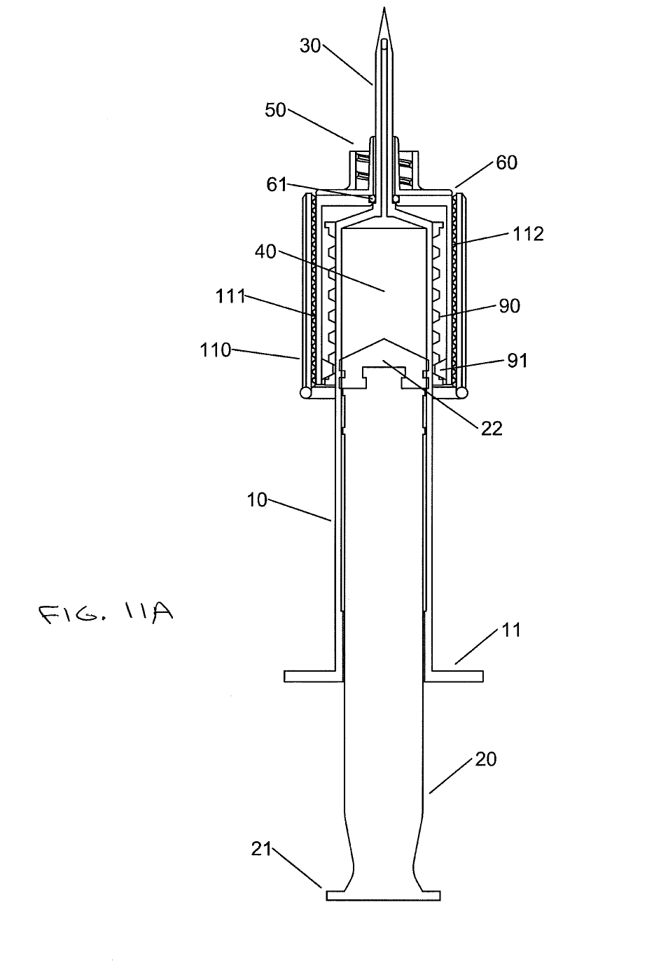

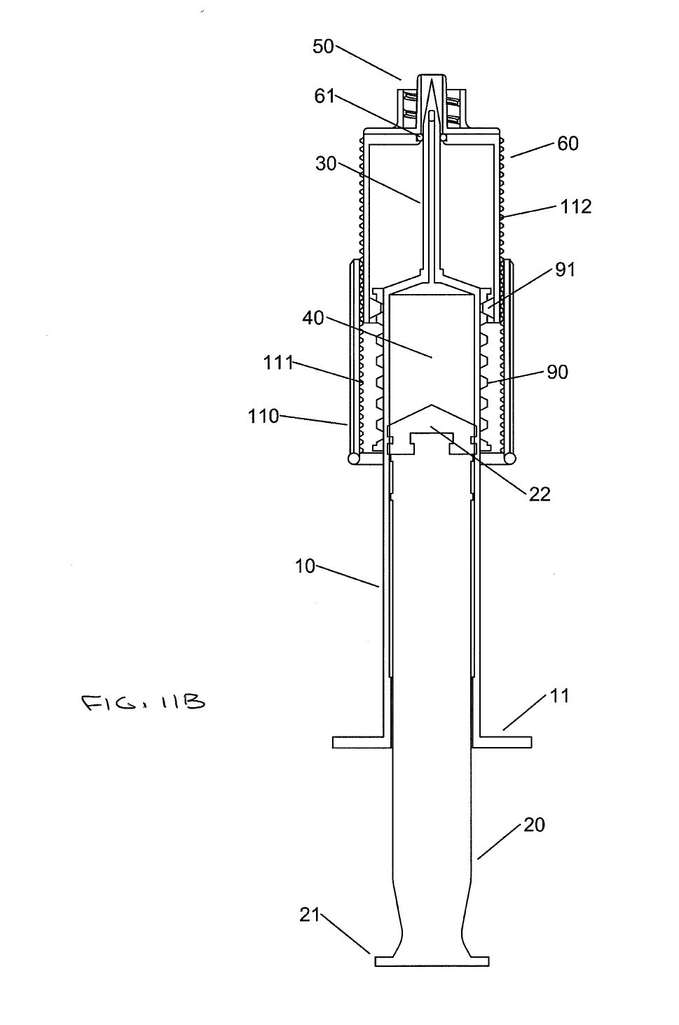

In yet another embodiment (FIGS. 11A and 11B), which is a variation of FIGS. 9A and 9B, there is an additional external cylinder 110 that has an internal thread 111, such that rotation of the cylinder 110 causes advancement of the collar 60 from the retracted position (FIG. 11A) to the advanced position (FIG. 11B) by the interface of the thread 111 with an external collar thread 112 and by the internal collar thread 90 interfacing with the protuberance 91.

The syringe system 1 may draw up medication from a medication bottle in a standard fashion with the collar 60 reversibly locked in its retracted position (FIG. 1A) and the operator pulling back on the proximal handle 21 of the plunger 20. By this action, medication is drawn into the enlarging interior space 40. Once this is complete, the collar 60 may be reversibly locked into its extended position as seen in FIG. 2, simultaneously covering the distal end 32 of the needle 30, and maintaining a seal between the sealing component 61 and the needle 30. After converting the system 1 from needled to needleless, the syringe system 1 may be easily attached to the needleless tubing system via, for example, the threads 57 on the fluid port 50. The plunger 20 may be advanced to move the medication distally through the sealed system and out through the needle openings 33 into the needleless tubing system.

The size and shape of the syringe and associated components will vary to suit a particular application and patient (e.g., adult or pediatric). The specific dimensions, capacities, configurations will be selected to suit a particular application. For example, the syringe can have a volumetric capacity of about 0.05 cc to about 100.0 cc, the needle can be from about 33 gauge to about 10 gauge, and the needleless interface can be a luer type fitting.

Generally, the components of the syringe system can be manufactured by injection molding or by modifying an extruded tube. For example, extrusion can be used to provide a uniform polymeric tube, to which other components are attached. Insert molding can be used to provide the desired geometry of the components and openings in a component can then be created in the desired locations as a subsequent mechanical operation. Additional manufacturing techniques include blow molding, compression molding, transfer molding, and any other molding techniques. For example, single-shot or multi-shot injection molding. The various components of the syringe system can be assembled by snap fitting, bonding, and/or tongue and groove connection.

The syringe and related components can be manufactured from glass or plastic and may be made of a biocompatible material, such as, for example, polyurethane, silicones, polyethylenes, nylons, polyesters and polyester elastomers, either with or without reinforcement. Stainless steel and titanium can also be used, for example, for the needle. In addition, the needle can be formed from a polymeric material or a combination of metal and polymeric materials, for example, the needle can be stainless steel with a polymer over-molded on to the needle. The needle can have a sharp or blunt tip. Also, the polymeric materials may be used in combination with other materials, for example, natural or synthetic rubber. Other suitable materials will be apparent to those skilled in the art. In one embodiment, the barrel of the syringe is made of plastic, has graduated marks indicating the volume of fluid in the syringe, and is substantially transparent. The syringe plunger or piston may be made of rubber, which provides a good seal between the piston and the barrel.

Various examples of syringe systems and their manufacturing, material, and arrangement details can be found in U.S. Pat. Nos. 7,182,734; 5,817,065; 5,681,295; and 5,273,543, the entire disclosures of which are incorporated herein by reference in their entireties.

Having described certain embodiments of the invention, it will be apparent to those of ordinary skill in the art that other embodiments incorporating the concepts disclosed herein may be used without departing from the spirit and scope of the invention. The described embodiments are to be considered in all respects as only illustrative and not restrictive.

What is claimed:

1. A syringe system comprising:
a syringe barrel defining an interior space;
a syringe plunger slidably disposed within the syringe barrel;
a needle coupled to a distal end of the syringe barrel and in fluid communication with the interior space;
a collar slidably disposed about the syringe barrel, the collar coupled to the syringe barrel to prevent inadvertent disengagement therefrom; and
a fluid port disposed at a distal end of the collar, the fluid port configured to interface with a needleless tubing system.

2. The syringe system of claim 1, wherein the collar is configured to slide along at least a longitudinal portion of the syringe barrel and can be secured in a first retracted position exposing the needle and a second advanced position encapsulating the needle and presenting the fluid port for interfacing with the needleless tubing system.

3. The syringe system of claim 1, wherein the fluid port comprises threads and is securable to the needleless tubing system by a rotation.

4. The syringe system of claim 1, wherein the fluid port is a luer lock type fitting.

5. The syringe system of clam 2, wherein the collar is lockable in at least one of the first position and the second position.

6. The syringe system of claim 5, further comprising a locking mechanism disposed on at least one of the syringe barrel and the collar for locking the collar in the second, advanced position.

7. The syringe system of claim 6, wherein the syringe barrel includes at least one slotted rail disposed on an outer surface of the syringe barrel and oriented longitudinally along a length of the syringe barrel and the collar includes at least one protuberance disposed on an inner surface of the collar for engaging the slotted rail, wherein the collar slides along the syringe barrel via engagement of the protuberance and the slotted rail.

8. The syringe system of claim 7, wherein the longitudinal rail includes a transverse portion extending from a distal end of the longitudinal rail, wherein the transverse portion of the rail allows guided, rotational movement of the collar when in the second position.

9. The syringe system of claim 8, wherein the locking mechanism is disposed on the transverse portion of the rail and the collar engages the locking mechanism when rotated in the second position.

10. The syringe system of claim 9, further comprising a second locking mechanism disposed near the proximal end of the longitudinal rail, the second locking mechanism configured to secure the collar in the first position.

11. The syringe system of claim 9, wherein the locking mechanism comprises a frictional engagement between the protuberance and the rail.

12. The syringe system of claim 9, wherein the locking mechanism comprises a projection that prevents the return rotational movement of the collar by blocking the protuberance.

13. The syringe system of claim 1, wherein the collar is guided between the first position and the second position by a thread disposed on an external diameter of the syringe barrel.

14. The syringe system of claim 1, wherein the fluid port is configured for attachment of a secondary needle.

15. The syringe system of claim 1, wherein the collar is biased in at least one of the first position and the second position.

16. The syringe system of claim 15, wherein the collar includes a spring mechanism for biasing the collar in the second position.

17. The syringe system of claim 1, wherein the fluid port conforms to at least one of ISO standard 594-1 and 594-2.

18. The syringe system of claim 1, wherein the collar is removable from the syringe barrel and interchangeable with a second collar with an alternative fluid port.

19. The syringe system of claim 1, wherein a distal portion of the collar comprises an internal channel for receiving at least a distal portion of the needle, wherein the needle moves through the channel when the collar is retracted into the first position.

20. The syringe system of claim 19, wherein the collar further comprises an O-ring disposed in an annular groove formed in an internal surface of the channel, the O-ring providing a seal between an outer diameter of the needle and an inner diameter of the channel for the passage of a fluid between the syringe barrel and the needleless tubing system.

21. The syringe system of claim 1, wherein the syringe barrel has a generally circular cross-sectional shape and the collar has a generally elliptical internal cross-sectional shape, thereby providing sufficient surface tension between the collar and syringe barrel to prevent sliding movement between the collar and the syringe barrel, the collar deformable by a light pressure to deform the internal cross-sectional shape of the collar from generally elliptical to generally circular and of a larger diameter than an external diameter of the syringe barrel to enable slidable movement therebetween.

22. A syringe system comprising:
a syringe barrel defining an interior space;
a syringe plunger slidably disposed within the syringe barrel;
a needle coupled to a distal end of the syringe barrel and in fluid communication with the interior space;
a collar threadedly engaged with the syringe barrel, the collar movable along the length of the syringe barrel by rotational movement thereof; and
a fluid port disposed at a distal end of the collar, the fluid port configured to interface with a needleless tubing system.
